(19) United States
(12) Patent Application Publication
(10) Pub. No.: US 2012/0178114 A1
(43) Pub. Date: Jul. 12, 2012

(54) METHODS AND DEVICES FOR DETECTING THROMBIN GENERATION

(75) Inventors: Whyte G. Owen, Rochester, MN (US); Daniel G. Ericson, Rochester, MN (US)

(73) Assignees: SUBC, INC., Rochester, MN (US); MAYO FOUNDATION FOR MEDICAL EDUCATION AND RESEARCH, Rochester, MN (US)

(21) Appl. No.: 12/672,973

(22) PCT Filed: Aug. 8, 2008
(86) PCT No.: PCT/US08/72555

§ 371 Date: Mar. 26, 2012

Related U.S. Application Data
(60) Provisional application No. 60/964,272, filed on Aug. 10, 2007.

(51) Int. Cl. G01N 21/76; C12M 1/34; C12Q 1/56

(52) U.S. Cl. 435/13; 435/287.1; 435/288.7

(57) ABSTRACT

Methods and devices for detecting thrombin generation are disclosed. Generally, the methods include combining a blood sample with a reagent composition so that reaction of the reagent composition and thrombin, if present in the sample, produces a detectable signal; and detecting the detectable signal. Generally, the devices include a fluid-tight material forming at least one passageway; a first chamber in fluid communication with at least one passageway; and at least one reagent disposed on a surface of or contained in either a chamber or a passageway. In some embodiments, the passageway is configured to permit capillary flow of fluid, while in other embodiments, fluid flow is accomplished through a pump functionally linked to at least one passageway. In some embodiments, the device may further include a signal detector positioned to detect a signal generated in a chamber or passageway. In certain embodiments, the device may further include a microprocessor functionally linked to the signal detector.

### CROSS-REFERENCE TO RELATED APPLICATION

This application claims priority to U.S. Provisional Patent Application Ser. No. 60/964,272, filed Aug. 10, 2007.

### BACKGROUND

Measurements of blood clotting can be useful for the management and diagnosis and management of bleeding disorders and drug therapy for thrombosis. Additionally, there is a growing demand for methodologies that define propensity, or risk, for thrombosis.

Gaining insight into hemostasis and thrombosis can be difficult without an approach that encompasses platelet function in concert with plasma proteins. Laboratory evaluation of platelet function in preclinical and clinical research settings has been based almost exclusively on measurements of platelet aggregation either in whole blood or in platelet-rich plasma—plasma from which red cells have been removed by centrifugation. These measurements can be operator intensive, semi-quantitative and, because of paracrine cooperativity, can have inadequate sensitivity and specificity for certain indications. Often, assays must be carried out within 2-3 hours of obtaining a blood sample and can require rigid quality control of sample procurement and processing, including controlling temperature and exposure to room air. In addition, standardization can be difficult.

Moreover, certain methods for verifying the efficacy of antithrombotic therapies can involve a patient visiting a secondary or tertiary medical center for evaluation. This level of inconvenience can reduce patient compliance.

### SUMMARY

The present invention provides a method of detecting thrombin in a sample. Generally, the method includes combining a blood sample with a reagent composition, wherein reaction of the reagent composition and thrombin, if present in the sample, produces a detectable signal; and detecting the detectable signal.

In some embodiments, the detectable signal is a chemiluminescent signal.

In some embodiments, a reagent composition includes a thrombin substrate. In certain embodiments, a reagent composition can include benzoylarginine ethyl ester, alcohol oxidase, and luminol.

In another aspect, the present invention provides a device for detecting thrombin in a sample. In one embodiment, the device includes a fluid-tight material forming at least one passageway; a first chamber in fluid communication with at least one passageway; at least one reagent disposed on a surface of or contained in either a chamber or a passageway; and a pump functionally linked to the at least one passageway. In an alternative embodiment, the device includes a fluid-tight material forming at least one passageway, wherein the passageway is configured to permit capillary flow of fluid; a first chamber in fluid communication with at least one passageway; and at least one reagent disposed on a surface of or contained in either a chamber or a passageway.

In some embodiments, the device can further include a signal detector positioned to detect a signal generated in a chamber or passageway. In some embodiments, the signal detector is a photodiode. In certain embodiments, the device further includes a photomultiplier. In certain embodiments, a microprocessor may be functionally linked to the signal detector.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 is a schematic diagram showing thrombin analysis according to an embodiment of the invention.

FIG. 2 is a line graph showing signal detection as a function of thrombin activity.

### DETAILED DESCRIPTION OF ILLUSTRATIVE EMBODIMENTS OF THE INVENTION

The invention includes methods and devices for detecting thrombin in a blood sample. Thus, the methods and devices may be used to help identify proper platelet function in the absence of platelet drug therapy and/or to identify inhibited platelet function in the presence of platelet drug therapy. Thus, the methods and devices can be useful for diagnosing blood clotting pathologies—either those conditions in which clots fail to form as they are supposed to (e.g., hemophilia), or conditions in which clots form at an inappropriate time and/or place (e.g., thrombosis). The methods and devices also may be used to monitor the effectiveness of clot modifying therapies. Because of the rapid analysis possible using the methods and devices, the methods and devices can have point of care utility.

“Blood” as used herein refers to whole blood or to a blood fraction containing platelets. Accordingly, the term “blood” includes platelet-containing plasma, purified platelets, or any blood fraction containing platelets. The term “whole blood” refers to blood that has not been fractionated.

“Platelet activator” refers to a substance that upon contact with platelets induces platelets to perform any platelet function without a requirement that the platelets be exposed to shear or any other mechanical activator.

“Platelet function” refers to any platelet activity including, for example, adhering to a substrate, changing shape, releasing chemical messengers or clotting factors stored in the cytoplasm of the platelets, and/or aggregating with other platelets, and combinations thereof.

“React,” “reaction,” “reactant,” and variations thereof refer to both catalytic and non-catalytic chemical transformations. Thus, a catalyst may be considered a reactant, and considered to react with a substrate even though the catalyst is itself unchanged by the reaction.

As used herein, “a,” “an,” “the,” “at least one,” and “one or more” are used interchangeably. Thus, for example, a reagent composition that comprises “a” reagent can be interpreted to mean that the reagent composition includes “one or more” reagents.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

In one aspect, the invention includes a method of detecting thrombin in a biological sample such as, for example, a blood sample. The method includes a coupled chemistry that enables thrombin generation to be detected in small samples. In some embodiments, thrombin generation is measured as chemiluminescence in a simple microfluidic photometer.

Thrombin (activated Factor II [IIa]) is a coagulation protein that has many effects in the coagulation cascade. It is a serine protease that converts soluble fibrinogen into insoluble strands of fibrin, as well as catalyzing many other coagulation-related reactions.

Assays based on clotting times of blood or plasma may not always be sensitive to subtle changes in blood that influence the propensity of blood to generate thrombin and, by extension, to initiate thrombosis. Such assays are limited by the formation of the fibrin clot as an endpoint, an event that occurs early in the clotting process as thrombin generation is just beginning.

There is growing evidence that information generated over the entire thrombin generation progress curve—e.g., after the fibrin clot is formed—may be useful for diagnosing thrombophilia. For example, the amount of thrombin generated in whole blood can be sensitive to subtle changes in platelet function. Also, the platelet contribution to thrombin generation in blood cannot be recapitulated with synthetic procoagulant membranes. Certain assays permit one to measure thrombin generated in platelet-rich plasma. These technologies can require anticoagulation of the blood sample. These technologies also can require either sub-sampling blood or preparing platelet-rich plasma, each of which can require exacting technical expertise.

In contrast, methods described herein measure the underlying biochemistry that creates a clot. As such, the methods may allow one to gather much more information and do so with a simpler blood sample. The methods measure the dynamics of thrombin generation, which both creates and controls the clotting process. To enable measurement of thrombin in whole blood—which often obscures chemical signals—thrombin may be assayed with a coupled chemistry that generates a detectable signal such as, for example, light. The reaction can take place in a chamber to which a signal detector (e.g., a photodetector) and a microprocessor (to analyze the signal) are functionally linked. The sample volume needed to perform the assay may be small enough that the blood can be obtained with a finger stick.

The blood sample may be collected by venipuncture with or without an anticoagulant or an attenuator such as, for example, a thrombin inhibitor (e.g., hirudin). In some embodiments, the subject whose platelet function is being monitored may be receiving treatment with an anti-platelet agent. In particular embodiments, a suitable anti-platelet agent can include, for example, a cyclooxygenase inhibitor (e.g., aspirin or other salicylates), an ADP inhibitor (e.g., clopidogrel (PLAVIX) and ticlopidine), a GPIIbIIIa inhibitor (e.g., tirofiban, eptifibatide, and abciximab), or a combination thereof.

The blood sample may be obtained from a subject and then analyzed without any processing and without the addition of any agents (e.g., anti-coagulants or platelet activators). Alternatively, the blood sample may be processed and the methods performed using any processed blood fraction that contains platelets such as, for example, a blood fraction enriched for platelets. Additionally and/or alternatively, a blood sample may have certain agents added to it.

In some embodiments, the method may include performing the assay using a suitable assay device. In such embodiments, the sample may be loaded into the assay device by any suitable method such as, for example, by capillary action or by using, e.g., a pump or syringe. The loaded sample may be combined with at least one reagent composition that includes one or more reagents. A reagent composition may include one or more reagents dissolved or suspended in a suitable buffer. Alternatively, a reagent composition may form a coating on a portion of a chamber or passageway of a suitable assay device. In other embodiments, a reagent composition may be provided as an area that includes one or more reagents immobilized to or incorporated into a passageway or chamber of a suitable assay device. In particular embodiments, a sample may be contacted with more than one reagent composition. In such embodiments, the regent compositions may be provided as a solution, a suspension, a coating, or an area as just described, or in any combination thereof. Methods of coating surfaces and immobilizing reagents are well known to those skilled in the art.

A reagent composition can include at least one component that can react with thrombin. Because thrombin is an enzyme that catalyzes many coagulation-related reactions, the component that can react with thrombin may be a substrate of thrombin catalytic activity. In some embodiments, a reagent composition can include a thrombin substrate that includes an alcohol leaving group so that catalysis by thrombin yields an alcohol. In one particular embodiment, a reagent composition can include benzoylarginine ethyl ester (BAEE). Other suitable thrombin substrates include for example, thrombin substrates containing alcohol esters. A reagent composition can include any combination of components that can react with thrombin.

A reagent composition can include at least one component that is capable of generating a detectable signal. In some embodiments, the component that is capable of generating a detectable signal may be the same component that can react with thrombin. However, in other embodiments, the component that can react with thrombin and the component that is capable of generating a detectable signal may be two separate components. For example, the sample may include a component that reacts with thrombin to form an intermediate. The intermediate may then react with another reagent of the reagent composition in order to generate the detectable signal.

The detectable signal may be any suitable signal such as, for example, a chemiluminescent label, a fluorescent label, a colorimetric label, an amperometric label, or a radiolabel. Suitable detectable signals are well known to those of skill in the art. In some embodiments, the detectable signal may be a chemiluminescent signal. In one such embodiment, a reagent composition includes luminol, which can react with hydrogen peroxide to produce a chemiluminescent signal. A chemiluminescent signal may be generated using any suitable reagent that can react with any chemical intermediate resulting from the reaction of thrombin and a component of the reagent composition. Hydrogen peroxide is one suitable intermediate, but other suitable intermediates include, for example, NADH.

In alternative embodiments, the signal may be an amperometric signal. For example, reagents that can react with hydrogen peroxide and generate an amperometric signal are commercially available and well know to those skilled in the art.

In some embodiments, the detectable signal may be detectable in real time. Thus, the detectable signal may be detected in less than about 10 minutes from the time that the sample is combined with the reagent composition. For example, the detectable signal may be detectable in less than about 5 minutes, less than about 2 minutes, less than about 1 minute, less than about 30 seconds, less than about 10 seconds, less than about 5 seconds, less than about 2 seconds, or less than about 1 second after the sample is combined with the reagent composition.

In some embodiments, a reagent composition may include one or more additional reagents. Such reagents may, in some embodiments, be reagents that are involved in chemical reactions necessary to produce the detectable signal. For example, in embodiments in which BAEE is a component of the reagent composition, catalysis of BAEE by thrombin produces ethanol. In some embodiments, the ethanol may be converted to hydrogen peroxide by alcohol oxidase. Subsequently, as noted above, hydrogen peroxide can react with an appropriate reagent to generate, for example, a chemiluminescent or amperometric signal.

In some embodiments, a reagent composition can include a platelet activator. The term “platelet activator” refers to a biological platelet activator or a chemical platelet activator. As used herein, a biological platelet activator refers to an agent found naturally in a mammalian body that has the biological role of activating platelets. Biological platelet activators include, for example, ADP, thrombin, thromboxane A₂, serotonin, and epinephrine. As used herein, a chemical platelet activator refers to a compound, other than a biological platelet activator, that activates platelets. Chemical platelet activators include, for example, non-biological synthetic compounds, derivatives of biological agents that activate platelets, biological agents found in plants or microorganisms that activate platelets, and the thrombin receptor activating peptide SFLLRN (SEQ ID NO:1). A platelet activator may be desired if, for example, the blood sample is obtained from a subject being treated with, for example, an ADP inhibitor.

In some embodiments, a reagent composition can include other compounds that are not platelet activators but are beneficial to clot formation such as, for example, fibrinogen, fibrin, and von Willebrand factor. Such a component may be desired if, for example, the blood sample is obtained from a subject being treated with, for example, a GPIIbIIIa inhibitor. In certain embodiments, for example, fibrinogen may be a preferred agent since it binds to the GPIIbIIIa receptors.

A reagent composition may include one or more anti-coagulants. An anti-coagulant in the reagent composition may prolong the amount of time a blood sample may be handled before being analyzed. In such embodiments, the anti-coagulant may be sequestered from reagents involved in the thrombin detection assay so that the blood sample can be mixed with the anti-coagulant and, therefore, stabilized for a time before the thrombin detection assay is performed. A blood sample mixed with an anti-coagulant may further enable pharmacological manipulation of the platelets in vitro in order to explore mechanisms of changes in platelet function associated with thrombotic disease. For example, a blood sample may remain stable for two to three hours after collection when the blood sample is mixed with hirudin, an absolutely specific inhibitor of thrombin, or with tick anticoagulant peptide, a factor Xa inhibitor. This represents an approximately 10- to 15-fold increase in the time that a blood sample may remain stable. Suitable anti-coagulants include, for example, hirudin, tick anticoagulant peptide, other specific clotting inhibitors or clotting enzymes, and combinations thereof.

A reagent composition may include one or more pro-coagulants. In such embodiments, the method may be suited for point of care clotting tests. A reagent composition that includes, for example, ecarin (a prothrombin activator from Echis carinatus venom) may be used to generate reproducible clotting data using either plasma or blood as an analyte. In some embodiments, a pro-coagulant may be dried (e.g., coated) or otherwise incorporated into a passageway or chamber so that a blood sample can dissolve the pro-coagulant as it is loaded. Suitable pro-coagulants include, for example, ecarin, Russell's viper venom, activated factor X, tissue factor, and combinations thereof.

In an exemplary embodiment, a blood sample is combined with benzoylarginine ethyl ester (BAEE) and a platelet activator such as ADP or the thrombin receptor activating peptide SFLLRN (SEQ ID NO:1). In certain embodiments, the sample may be agitated or mixed, thereby promoting contact between platelets in the sample.

As thrombin activity develops, it catalyzes hydrolysis of the BAEE, which yields ethanol. As the ethanol is generated it is oxidized by excess alcohol oxidase to yield hydrogen peroxide, which reacts with excess luminol to yield light (hv). The light is detected using a photodetector with optional signal amplification.

Thrombin in blood clotted with trace tissue factor peaks at about 100 nM (about 7% of the starting prothrombin concentration) with a peak width of 20 minutes. The Kₘ for BAEE is 100 μM and k_{cat} is 50 s⁻¹, so about 50 μM BAEE would be consumed if BAEE is provided in the assay at an initial concentration of 100 μM, and the rate would fall by half by the end. Thus, without attenuation by endogenous substrates, there is excess BAEE capacity at a concentration that does not substantially compete with thrombin for other substrates. The luminescence progress curve does not yield an absolute thrombin activity, but the assay can be calibrated with a primary standard of guanidinobenzoyl-thrombin, a transiently inactive derivative which, when added to blood, reactivates with predictable kinetics.

The methods described above may be performed using any suitable device. In some embodiments, generally, a suitable device can include a microfluidic system in which various reagents are loaded as separate zones. The device can include at least one passageway and at least one chamber in fluid communication with the at least one passageway. The passageway may be configured to accept delivery of a sample—e.g., via an inlet port. A reagent composition may be disposed on a surface or otherwise contained in either a passageway or chamber. The reagent composition includes at least one reagent (including, e.g., a combination of reagents) capable of generating a detectable signal when contacted with at least a portion of a biological sample containing thrombin. For example, as described above, a reagent composition may be provided as a solution or suspension that could be contained within a portion of a passageway or chamber. Alternatively, a reagent composition may be provided as a coating disposed on a surface of a passageway or chamber. In other cases, a reagent composition can be provided as an area to which one or more reagents is immobilized to or incorporated into the material from which the device is constructed. The device may further include a pump in fluid communication with at least one passageway. The pump may be used to control fluid flow within the device. Alternatively, fluid flow may be controlled by capillary action resulting from the configuration and dimensions of the at least one passageway. The device may further include a signal detector designed to detect the signal generated upon contact of a portion of the sample with the reagent composition.

In such a system, for example, a reservoir (including, e.g., a chamber or passageway) may be filled with a simple carrier or buffer. Next, a sample zone may be formed by drawing a volume of sample into the reservoir. Finally, one or more reagent zones may be drawn into the reservoir. In this way, it is possible to construct a stack of well defined zones that can be mixed together to generate a detectable species.

For a thrombin generation assay, a series of stacked zones of, for example, BAEE, alcohol oxidase, and luminol may be loaded into a reservoir of a suitable assay device. Each zone may be separated by an air interface. In one particular embodiment, the total collective volume of the BAEE, alcohol oxidase, and luminol preloaded zones may be, for example, 10 microliters (μL). A volume of blood sample (e.g., 40 μL) may then be drawn into the reservoir. The blood sample may be obtained, for example, from a finger stick and drawn directly into a capillary tube connected to a dedicated valve. Once all of the zones are formed (which may take less than six seconds), the zones may be mixed to generate a detectable signal. If necessary, the detectable signal may be detected and/or quantified using an instrument suitable for detecting and/or quantifying the detectable signal. Zone mixing and signal detection may take less than one second.

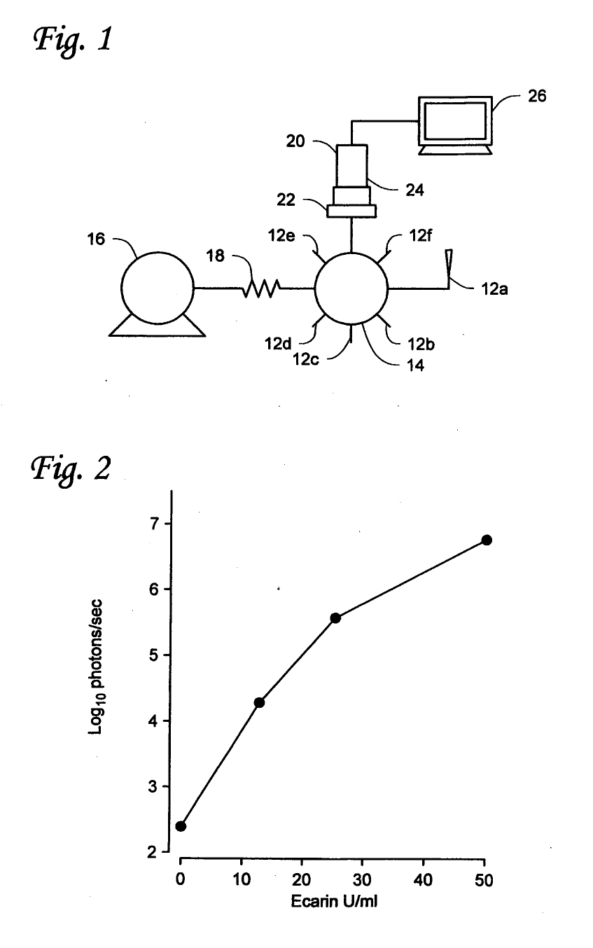

In certain embodiments, the device can include a microfluidic system (SUBC Inc., Rochester, Minn.) in which the various reagents are loaded as separate zones (FIG. 1). Such a system may use, for example, microsyringes 12a-f for sample and reagent delivery, a multiport valve 14 for sorting reagents, and a photodetector 20 for output. The photodetector 20 can include a cell 22 for detection by photocounter 24. The photocounter 24 may be connected to a computer 26 for data analysis and storage. The device may include a pump 16 that may be used to control the flow of fluids. The device may further include a mixing coil 18 in which reagents may be mixed. A pump 16, if present, can allow bidirectional control of fluid flow through passageways that provide fluid communication between system components.

In certain embodiments, one or more components of the device may be housed in a cartridge. Such a device may include a fluid-tight material that defines at least one passageway and at least one chamber in fluid communication with at least one passageway. The cartridge can include a single channel, preferably accommodating, for example, about 20 μL of blood, or dual channels, preferably accommodating, for example, a collective total of 40 μL of blood. The cartridge may be designed to accept a common 75 millimeters (mm) capillary tube which may be connected to cartridge in any suitable manner such as, for example, bonded into the cartridge using, for example, a common adhesive. Alternatively, the cartridge may be designed to temporarily accept, for example, a capillary tube or syringe needle for delivery of sample or a reagent solution or suspension. A main channel of the cartridge (or the only channel in a single-channel cartridge) can be any suitable dimensions such as, for example, approximately 0.051 centimeters (cm) deep by approximately 0.089 cm wide. The main channel can be used to transport a blood sample to a chamber located within the main channel. The chamber may be preloaded with one or more reagents or, as described above, reagents may be added to the cartridge sequentially.

In some embodiments, the device can include a restriction channel that creates shear stress within the blood sample, which in turn will activate the platelets. Mechanical activation of the platelets using a restriction channel can eliminate having to include a platelet activator in the reagent composition. The restriction channel area can be of any suitable dimensions such as, for example, approximately 0.025 cm deep by approximately 0.025 cm wide by approximately 0.20 cm long.

The cartridge may be manufactured from any suitable material such as, for example, polycarbonate, polyester, acrylic, and polystyrene. In certain embodiments, the cartridge may be made from polystyrene as the base material.

Some embodiments may include one or more reagent chambers in fluid communication with a mixing chamber. In such an embodiment, the blood sample may be introduced and transferred to the mixing chamber. The one or more reagent chambers may be preloaded with reagent composition or, alternatively, reagent composition may be directed into the one or more reagent chambers by controlled fluid flow (e.g., by use of pump and/or valve). The device may further include a diffusion barrier between a reagent chamber and the mixing chamber. The diffusion barrier may be formed from any suitable material such as, for example, a standard cellophane dialysis membrane. The diffusion barrier provides some level of control over the reaction rate as it controls entry of the reagents into the mixing chamber.

Certain embodiments do not include a pump, but instead rely on capillary flow for fluid transport.

The following discussion describes exemplary embodiments of the devices and methods described herein. The particular materials, the amounts used, the mixing times, as well as other conditions and details are exemplary. Alternative embodiments may be practiced using different materials, different amounts of materials, different mixing times, and different conditions.

The assay device can include a sample cell that may be, for example, about 50 μm thick and may accommodate, for example, a sample volume of approximately of 14 μL. A blood sample (e.g., whole blood) may be introduced into the device using microsyringe 12a. A pro-coagulant such as, for example, ecarin may be introduced into the device through microsyringe 12e. Pump 16 may draw the sample and the pro-coagulant through the multiport valve 14 and into the mixing coil 18 for mixing (e.g., approximately 20 seconds). BAEE may be introduced into the device through microsyringe 12c, drawn through the multiport valve 14 by pump 16 and into the mixing coil 18, where it is combined and mixed with the reaction mixture (e.g., for approximately 10 seconds). Alcohol oxidase may be introduced into the device through microsyringe 12d, drawn through the multiport valve 14 by pump 16 and into the mixing coil 18, where it is combined and mixed with the reaction mixture (e.g., for approximately 10 seconds). Luminol may be introduced into the device through microsyringe 12f, drawn through the multiport valve 14 by pump 16 and into the mixing coil 18, where it is combined and mixed with the reaction mixture (e.g., for approximately one second). Additional reagents, if desired, can be introduced into the device through microsyringe 12b and additional microsyringes, if present, drawn through the multiport valve 14 by pump 16 and into the mixing coil 18 at a time appropriate for the reagent, where it is mixed with the reaction mixture. Pump 16 transfers the reaction mixture through the multiport valve 14 into the cell 22.

The methods and devices described herein may have utility for several applications. The methods and devices can be used, for example, to monitor the effectiveness of anti-platelet agents in patients treated with anti-platelet agents. Such patients include, for example, those treated using interventional cardiology catheterization procedure such as, for example, angiograms, angioplasty, and stent placement. In addition, the methods can be used to monitor the effectiveness of anti-platelet agents in patients who, for example, have received an artificial heart valve.

Such a method can involve obtaining a test sample from a patient at a time point after the patient has been administered an anti-platelet agent. The test sample may be combined with a reagent composition in order to generate a detectable signal if thrombin is present in the test sample. The resulting signal may be compared to an appropriate reference signal and any differences between the test sample signal and the reference signal determined. Depending upon the particular reagents used, the signals (i.e., sample signal and reference signal) may by qualitative and/or quantitative in nature. The reference signal may include one or more standards recognized by those skilled in the art as indicative of specific and/or relative anti-platelet activity. Alternatively, the reference signal may be generated by combining the reagent composition with at least a portion of a reference sample from the patient. The reference sample may be obtained from the patient prior to having an anti-platelet agent administered (e.g., so that the reference signal may provide a baseline value). Alternatively, the reference sample may be obtained after the ant-platelet agent is administered to the patient but before the test sample is obtained from the patient (e.g., so that the time course of ant-platelet agent activity can be studied).

For example, the methods and devices can be used to monitor the effectiveness of an anti-platelet agent (e.g., aspirin) in patients taking the agent to prevent a cardiovascular event such as, for example, coronary thrombosis (e.g., heart attack), pulmonary embolism, stroke, or deep vein thrombosis due to excessive platelet activity. For example, aspirin is routinely administered in the ER when a patient is admitted with chest pain. The onset of the aspirin effects on platelets may be highly dose dependent and highly variable among individuals for a given dose, even when administered intravenously. The aspirin effects may be monitored in time by repeated performance of the method described herein over predetermined time intervals. Such a point of care measurement of a patient's response to aspirin would enable more rapid determination of whether an alternative dosage of anti-platelet agent or an alternative therapy may be indicated.

As another example, a patient may be tested, for example, prior to a surgical or dental procedure to determine whether the patient may be at risk for excessive bleeding during the procedure. The test signal may be compared to one or more standard reference signals in order to determine a patient's risk of excessive bleeding. If a patient is identified to be at risk of excessive bleeding, appropriate precautions can be taken such as, for example, performing the procedure in a setting where a blood transfusion or platelet transfusion is available.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Example 1

BAEE, alcohol oxidase, and luminol are loaded as discrete zone segments into the holding coil of a detection device. Each segment is separated by an air interface. The total volume of the BAEE/AO/Luminol preloaded zone segments is 10 μL. 40 μL of blood sample obtained from a finger stick is drawn directly into a capillary tube connected to a dedicated valve of the assay device. Stacking of all of the zones was accomplished in less than about six seconds. After the zone segments are stacked, the zone segments are mixed and advanced to a position directly in front of the photon counter. Zone mixing and fluid advancement were accomplished in less than one second.

### Example 2

A blood sample was combined as described below with a reagent composition that included BAEE, alcohol oxidase, luminol, and ecarin.

The assay was performed using a photometer (SUBC, Inc., Rochester, Minn.) that uses microsyringes for sample and reagent delivery, a multiport valve for sorting reagents, and a photomultiplier for output.

The sample cell of the photometer is 50 μm thick and uses a minimum of 14 μL of sample. Ecarin and the whole blood sample were mixed for 20 seconds. BAEE was added and mixed for 10 seconds. Alcohol oxidase was added and mixed for 10 seconds. Luminol was added and mixed for 1 second. Finally, the mixture was transferred to the photomultiplier/photon counter for analysis. From a background of 253 photons/sec, a signal as high as 24,000 times background was generated (FIG. 2).

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1. A method of detecting thrombin in a biological sample, the method comprising:
combining a biological sample with a reagent composition that produces a detectable signal when contacted with at least a portion of a sample containing thrombin; and
detecting the detectable signal.

2. The method of claim 1 wherein the reagent composition comprises a thrombin substrate, and wherein reaction of the reagent composition and thrombin comprises forming an intermediate.

3. The method of claim 2 wherein the reagent composition further comprises a reagent that reacts with the intermediate.

4. The method of claim 2 wherein the reagent composition comprises:
an enzyme that further reacts with intermediate, thereby producing a second intermediate; and
a compound that reacts with the second intermediate to generate a detectable signal.

5. The method of claim 4 wherein the compound that reacts with the second intermediate comprises luminol.

6. The method of claim 1 wherein the thrombin substrate comprises an alcohol leaving group.

7. The method of claim 6 wherein the thrombin substrate comprises benzoylarginine ethyl ester.

8. The method of claim 6 wherein the reagent composition comprises alcohol oxidase.

9. The method of claim 8 wherein the reagent composition further comprises a compound that reacts with hydrogen peroxide to generate a detectable signal.

10. The method of claim 9 wherein the compound comprises luminol.

11. The method of claim 1 further comprising amplifying the detectable signal.

12. The method of claim 1 wherein the detectable signal is a chemiluminescent signal.

13. The method of claim 1 wherein the detectable signal is detected in real time.

14. The method of claim 2 wherein the reagent composition comprises a platelet activator.

15. The method of claim 2 wherein the reagent composition comprises a platelet inhibitor.

16. The method of claim 1 further comprising activating platelets in the biological sample.

17. The method of claim 16 wherein platelets are activated by contacting the sample with a platelet activator.

18. The method of claim 16 wherein platelets are activated by passing the biological sample through a passageway having a restriction channel configured to produce sufficient shear in a biological sample passed through the restriction to activate the platelets.

19. A device for detecting thrombin in a sample comprising:
a fluid-tight material forming at least one passageway;
a first chamber in fluid communication with at least one passageway;
at least one reagent disposed on a surface of or contained in either a chamber or a passageway, wherein the at least one reagent generates a detectable signal when combined with at least a portion of a sample containing thrombin; and
a pump functionally linked to the at least one passageway.

20. A device for detecting thrombin in a sample comprising:
a fluid-tight material forming at least one passageway, wherein the passageway is configured to permit capillary flow of fluid;
a first chamber in fluid communication with at least one passageway; and
at least one reagent disposed on a surface of or contained in either a chamber or a passageway.

21. The device of claim 20 further comprising a pump functionally linked to at least one passageway.

22. The device of claim 20 further comprising a signal detector positioned to detect a signal generated in a chamber or passageway.

23. The device of claim 22 further comprising a microprocessor functionally linked to the signal detector.

24. The device of claim 20 wherein at least one chamber contains a thrombin substrate.

25. The device of claim 20 further comprising at least one restriction in at least one passageway, wherein the restriction is configured to generate sufficient shear in a blood sample passed through the restriction to activate platelets in the blood sample.

26. The device of claim 20 further comprising at least one additional chamber in fluid communication with at least one passageway and the chamber containing the thrombin substrate.

27. The device of claim 26 wherein the at least one additional chamber comprises a mixing chamber, and further comprising a diffusion bather between the first chamber and the mixing chamber.

28. The device of claim 27 wherein the diffusion barrier comprises a dialysis membrane.

29. The device of claim 20 wherein the signal comprises a chemiluminescent signal and the signal detector comprises a photodetector.

30. The device of claim 29 further comprising a photomultiplier.

31. The device of claim 29 further comprising an optically transparent interface between the photodetector and at least a portion of a chamber or passageway in which the chemiluminescent signal is generated.

32. The device of claim 20 wherein at least one reagent is a component of a solution contained in a portion of a chamber or a portion of a passageway.

33. The device of claim 20 wherein at least one reagent is incorporated into a coating of at least one chamber or at least one passageway.

34. The device of claim 20 wherein at least one reagent is immobilized to a surface of at least one chamber or at least one passageway.

35. A method of monitoring an anti-platelet agent in a patient, the method comprising:
obtaining a biological sample from a patient at a first time point, wherein the first time point is a time after administration of an anti-platelet agent to the patient;
combining at least a portion of the biological sample with a reagent composition that produces a detectable signal when contacted with at least a portion of a biological sample containing thrombin;
detecting the detectable signal;
comparing the detectable signal to a reference signal, wherein a difference between the detectable signal and a reference signal indicates a difference in activity of the anti-platelet agent.

36. A method of monitoring the effectiveness of an anti-platelet agent, the method comprising:
obtaining a biological sample from a patient at a first time point, wherein the first time point is a time after administration of an anti-platelet agent to the patient;
combining at least a portion of the biological sample with a reagent composition that produces a detectable signal when contacted with at least a portion of a biological sample containing thrombin;
detecting the detectable signal;
comparing the detectable signal to a reference signal, wherein a difference between the detectable signal and a reference signal indicates effectiveness of the anti-platelet agent.

37. The method of claim 35 wherein comparing the detectable signal with a reference signal comprises:
obtaining a reference biological sample from a patient at a second time point, wherein the second time point is a time point before or after the first time point;
combining at least a portion of the reference biological sample with a reagent composition that produces a reference signal when contacted with at least a portion of a reference biological sample containing thrombin; and
detecting the reference signal.

38. A method of assessing a patient for risk of excessive bleeding, the method comprising:
obtaining a test value, comprising
obtaining a biological sample from a patient;
combining at least a portion of the biological sample with a reagent composition that produces a detectable signal when contacted with at least a portion of a biological sample containing thrombin; and
detecting the detectable signal; and
comparing the test value with a reference value, wherein a test value that is less than the reference value indicates that the patient is at risk of excessive bleeding.

39. The device of claim 19 further comprising a signal detector positioned to detect a signal generated in a chamber or passageway.

40. The device of claim 39 further comprising a microprocessor functionally linked to the signal detector.

41. The device of claim 19 wherein at least one chamber contains a thrombin substrate.

42. The device of claim 19 further comprising at least one restriction in at least one passageway, wherein the restriction is configured to generate sufficient shear in a blood sample passed through the restriction to activate platelets in the blood sample.

43. The device of claim 19 further comprising at least one additional chamber in fluid communication with at least one passageway and the chamber containing the thrombin substrate.

44. The device of claim 43 wherein the at least one additional chamber comprises a mixing chamber, and further comprising a diffusion barrier between the first chamber and the mixing chamber.

45. The device of claim 44 wherein the diffusion barrier comprises a dialysis membrane.

46. The device of claim 19 wherein the signal comprises a chemiluminescent signal and the signal detector comprises a photodetector.

47. The device of claim 46 further comprising a photomultiplier.

48. The device of claim 46 further comprising an optically transparent interface between the photodetector and at least a portion of a chamber or passageway in which the chemiluminescent signal is generated.

49. The device of claim 19 wherein at least one reagent is a component of a solution contained in a portion of a chamber or a portion of a passageway.

50. The device of claim 19 wherein at least one reagent is incorporated into a coating of at least one chamber or at least one passageway.

51. The device of claim 19 wherein at least one reagent is immobilized to a surface of at least one chamber or at least one passageway.

52. The method of claim 36 wherein comparing the detectable signal with a reference signal comprises:
obtaining a reference biological sample from a patient at a second time point, wherein the second time point is a time point before or after the first time point;
combining at least a portion of the reference biological sample with a reagent composition that produces a reference signal when contacted with at least a portion of a reference biological sample containing thrombin; and
detecting the reference signal.
